SEQUENCE LISTING (2 sequences)

SEQ ID NO: 1 - protein, 243 aa, Homo sapiens
  MISC_FEATURE (1)..(243): Sarcospan polypeptide
MGKNKQPRGQQRQGGPPAADAAGPDDMEPKKGTGAPKECGEEEPRTCCGCRFPLLLALLQLALGIAVTVVGFLMASISSSLLVRDTPFWAGIIVCLVAYLGLFMLCVSYQVDERTCIQFSMKLLYFLLSALGLTVCVLAVAFAAHHYSQLTQFTCETTLDSCQCKLPSSEPLSRTFVYRDVTDCTSVTGTFKLFLLIQMILNLVCGLVCLLACFVMWKHRYQVFYVGVRICSLTASEGPQQKI

SEQ ID NO: 2 - DNA, 2707 bp, Homo sapiens
  misc_feature (1)..(2707): cDNA transcript of mRNA
ggcaggccgagccagccgtgcgccgcgctccagggcccagggcgccgcacacgcacccacccacccacccagcctcgcagcgccatgggcaagaacaagcagccacgcggccagcagaggcaggggggcccgccggccgcggacgccgctgggcccgacgacatggagccgaagaagggcacgggggcccccaaggagtgcggggaggaggagccccggacctgctgcggctgccggttcccgctgctgctcgccctgctgcagctggccctgggcatcgccgtgaccgtggtgggcttcctcatggcgagcatcagctcctccctgctagtcagggacactccattttgggctgggatcattgtctgcttagtggcctatcttggcttgtttatgctttgtgtctcatatcaggttgacgaacggacatgtattcaattttctatgaaactgttatactttctgctgagtgccctgggcctgacggtctgtgtgctggccgtggcctttgccgcccaccactattcgcagctcacacagtttacctgtgagaccacactcgactcttgccagtgcaaactgccctcctcggagccgctcagcaggacctttgtttaccgggatgtgacggactgtaccagcgtcactggcactttcaaactgttcttactcatccagatgattcttaatttggtctgcggccttgtgtgcttgttggcctgctttgtgatgtggaaacataggtaccaggtcttctatgtgggtgtcaggatatgctccctcacggcttccgaaggcccccagcaaaagatctaacattcttgctcaaagttgcgagagaaagtagcacatggagtagctgaggttaaacaaacaaaaaaaaattttaaacaaagaaaggaaaaaaattgacaataaaagtcactcttctaattgaatatttttatatttttatgaaacaaaagagcatttcttcaggtttctattgtattttttttaacattcttgcagagaaagcaagatccaaattgattttgggatattaaaagttaacagaacactgaacaaggaaagaatggcatagatctatctttacagtctggagttaattcctgttaactcattttatccattccttacataatcttctttcctgttagtccagtttgatggtgtgaatggtgaatttcaggcccagttgctaaattttgtggcatcttcctctagtccttcccacctccagtcatcagccccactctgtcttggagacaggcaggaggtgggggaagagctgaatctctttattttccctggtagagacatcttcaaggcatgaaatagcttaaagagcagagtagaaatggaagaggctttgcaaaaggctagataactaacaacacctgggttggggcggcggcctcttctcttcagctcccttagcttggctccgtaagtggatcacttgccaaatgctttagatgattgcctctcaataattgaaaggtggtggtagttgtattctaaatgatgtagaaggtttaaaaataattacattatgcttctattctatcatctaaaacaaatcattaaaactaatttctagctaattgttaattataattatgctcagaagtctatttaatgagctctgactgtacttacgctgcactgtcggtgttaagagaaattactctcacaagagcagaggcctgaagattctttcttctgaaagccaagcaccacaaggaaaaaaaaattattaatagctcaggttaaaaacacccatttaaacaaaaacaagagcatttgtaataggaagtgtttatacaaacagcacatttgtgatatgttgaaaagcatctctcttggcaaccaatctatgtttgaggaagattgggtaatgctgatgtgttccattcatgaaactgtatttgatacataatcctattattaattcgtatgcttagtcaacctaggaaatcaaaataatgttttgaagttcttatttgagcaatatggccttgacttggagggtagttttagttgttttgtttttaagtgactgtggtttaaagcacaaatgccccaaggtggggagacttctctctgtgattattgttgctattaaattctgaactgtatccatattttaaggaaggagctaaaaatggaaattcatgaaacataaatggtatcaagaactttatcagtatgctttgttgaaagcagaaattaagataataattgagttcaattcgcctctccgcattgcctattgatacactttactaatcatgaaattctaacctaaaaggaaaacattttcctgcttgtcttagaagaaagtggaataattccactgattgtgataatggtttcaatttctacacaatataaatatccagtataaaggaaagcgttaagtcggtaagctagaggattgtaaatatcttttatgtcctctagataaaacacccgattaacagatgttaaaccttttaatgttttgatttgctttaaaaatggccttcctacacattagctccagctaaaaagacacattggagagcttagaggataagtctctggagcagaatttatcacacacaaaagttacaccaacagaataccaagcagaatgatgaggacctgtaaaataccttgtgccctattaaaaaaaaaaaaaaaaaaaaaaaaaaaaaaaaaa

(19) United States
(12) Patent Application Publication
(10) Pub. No.: US 2008/0221027 A1
(43) Pub. Date: Sep. 11, 2008

(54) Composition and Methods for the Treatment of Duchene Muscular Dystrophy

(75) Inventors: Rachelle H. Crosbie, Hollywood, CA (US); Angela K. Peter, San Diego, CA (US)

Correspondence Address: FISH & ASSOCIATES, PC;ROBERT D. FISH, 2603 Main Street, Suite 1050, Irvine, CA, 92614-6232, US

(73) Assignee: THE REGENTS OF THE UNIVERSITY OF CALIFORNIA, Oakland, CA (US)

(21) Appl. No.: 11/852,008

(22) Filed: Sep. 7, 2007

Related U.S. Application Data
(60) Provisional application No. 60/894,145, filed on Mar. 9, 2007.

(51) Int. Cl. A61K 38/00; C07K 16/00; A61K 31/70; C12N 15/00

(52) U.S. Cl. 514/12; 530/350; 514/44; 435/320.1

(57) ABSTRACT

Patients are treated for muscular dystrophy by artificially increasing effective sarcospan activity in the patient's muscle tissue. Sarcospan (or fragments thereof) can be administered directly to the tissue, or their production can be induced by gene therapy. Modifications are also contemplated to be effective, including for example, a lipid, TAT or other tag. Also contemplated are compositions that include a protein, mRNA or gene stabilizer, degradation inhibitor, and/or oligomerization inhibitor. Increased presence of sarcospan can also be accomplished indirectly, using a vector having an entire sarcospan gene sequence, fragment or related construct, modulating a transcription factor, and so forth.

This application claims priority to provisional application Ser. No. 60/894,145 filed Mar. 9, 2007.

### FIELD OF THE INVENTION

The field of the invention is muscular dystrophy.

### BACKGROUND

Muscular dystrophy is not a single disease, but a grouping of nine inherited disorders that include Duchenne's muscular dystrophy (DMD), Becker's muscular dystrophy, limb-girdle muscular dystrophy, acioscapulohumeral muscular dystrophy, and myotonic dystrophy. All of the muscular dystrophies are characterized by progressive weakness and wasting of the muscles.

DMD, the most common and severe type of muscular dystrophy, involves primary mutations in the dystrophin gene. The currently accepted theory is that these mutations cause the loss of dystrophin protein, and concomitant loss or dysfunction of the entire dystrophin-glycoprotein complex (DGC) from the sarcolemma (an extensible membrane enclosing the contractile substance of a muscle fiber).

There are currently no cures for Duchenne muscular dystrophy. Treatment is aimed merely at managing symptoms to improve the quality of life. By age 10, braces may be required for walking, and by age 12 most patients are confined to a wheelchair. Boys with DMD rarely live past age 20.

Gene therapy is a possibility, but progress has been hampered by the enormous size of the dystrophin gene. Nevertheless, U.S. Pat. No. 6,001,816 to Morsey (December 1999) describes in vivo muscle gene transfer of full-length dystrophin (although not for muscular dystrophy), and Mirus (www.mirusbio.com) is currently developing its MyoDys® plasmid DNA encoding the full-length human dystrophin gene. Mirus' Pathway IV™ delivery technology is used to administer the pDNA to a patient's limb skeletal muscles. U.S. Pat. No. 7,001,761 to Xiao (February 2006) describes efforts in a slightly different direction, utilizing dystrophin minigenes in the treatment of DMD and Becker muscular dystrophy. These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

An alternative avenue of potential treatment involves the sarcoglycans, which are a group of single pass transmembrane glycoproteins (α-, β-, γ-, and δ-sarcoglycan) that cause autosomal recessive limb girdle muscular dystrophies (Bonnemann et al., 1995; Jung et al., 1996; Lim et al., 1995; McNally et al., 1994; Nigro et al., 1996; Roberds et al., 1993; Roberds et al., 1994). US 2001/0029040 to Toyo-Oka (publ. October 2001) describes administering a gene expression vector to restore α, β, γ- and δ-sarcoglycan components in a living body, and early in 2007, a Muscular Dystrophy Association supported trial received a go-ahead to test a gene therapy to treat a deficiency of the alpha-sarcoglycan protein.

Another avenue of possible treatments involves utrophin, a protein analogue to dystrophin. In 1992 Matsumara et al. suggested that upregulation of utrophin could compensate for loss of dystrophin (Matsumura et al., 1992). Later the theory was put forth that if utrophin could functionally replace dystrophin, then it might be possible to upregulate utrophin expression in Duchenne muscular dystrophy patients (Matsumura et al., 1992; Tinsley et al., 1996, 1998). Since that time there have been numerous suggestions on ways to upregulate utrophin. (Krag 2001).

In 1997 Crosbie discovered sarcospan, a protein that interacts with the sarcoglycans to form a tight subcomplex within the DGC called the sarcoglycan-sarcospan subcomplex. In 1999 Crosbie et al. showed that sarcospan's localization to the sarcolemma is compromised in dystrophin and utrophin double null mice, which led to investigation of possible relationships between sarcospan and utrophin. (Crosbie et al. 1999). It turned out, however, that sarcospan-deficient animals do not develop an early-onset muscular dystrophy phenotype, and that sarcospan is not restored to the sarcolemma even in AR-LGMD cases with partial sarcoglycan deficiency where three out of four sarcoglycans are expressed (Crosbie et al., 1999). Moreover, no primary mutations in the sarcospan gene have ever been associated to date with any human disorder, despite extensive genetic studies (Crosbie et al., 2000; O'Brien et al., 2001). U.S. Pat. No. 6,207,878 to Campbell et al. (March 2001) suggested that sarcospan might be useful in treating obesity, but those suggestions were not borne out by later evidence. Thus, there is no current indication that modulation of sarcospan would have any positive effect on any disease, including any of the muscular dystrophies.

In short, there are still no cures on the immediate horizon for DMD. Although the current treatments under investigation may well turn out to be successful, the goals of those treatments are merely the delay and prevention of further loss of muscle function. Thus, there is still a need for new avenues of research and new treatments for muscular dystrophy and other muscle wasting diseases.

### SUMMARY OF THE INVENTION

The present invention provides compositions and methods in which a patient is treated for a disease involving diminution or dysfunction of a dystrophin-related complex by administering a composition to artificially increase sarcospan activity in a muscle tissue of the patient. Diseases of particular interest are muscular dystrophy and muscular atrophy.

In one class of embodiments the activity of sarcospan is increased by directly providing additional sarcospan, and/or or a sarcospan fragment, in the administered composition. Fragments of all practical lengths are contemplated, including especially fragments having lengths of at least 25, at least 50 or at least 100 amino acids.

In another class of embodiments the activity of sarcospan is increased by including in the composition an artificially modified polypeptide, again including at least a 25, 50 or 100 amino acid sequence of a sarcospan protein. Contemplated modifications include a lipid and/or a TAT or other tag. Also contemplated are compositions that include a protein, mRNA or gene stabilizer, degradation inhibitor, and/or oligomerization inhibitor.

In another class of embodiments the activity of sarcospan is increased indirectly, by including in the composition a transfection vector having an entire sarcospan gene sequence and/or a construct having at least a 30-mer, at least a 50-mer, or at least a 100-mer fragment of a sarcospan gene sequence. In yet another class of embodiments the composition comprises a transcription factor that upregulates expression of sarcospan protein.

The various described compositions can be administered in any suitable manner, including, for example, intravenously, intramuscularly, etc.

Various objects, features, aspects and advantages of the inventive subject matter will become more apparent from the following detailed description of preferred embodiments, along with the accompanying drawings in which like numerals represent like components.

### BRIEF DESCRIPTION OF THE DRAWING

FIG. 1 is a series of blots showing SSPN over-expression increasing expression of UGC in mdx mice.

FIG. 2 is a series of histology slides showing SSPN stabilizing the UGC at the sarcolemma.

FIGS. 3A and 3B are histology slides of quadriceps muscle showing SSPN-Tg:mdx mice displaying near normal muscle pathology.

FIG. 3C is a graph showing SSPN-Tg:mdx mice displaying a 3-fold reduction in central nuclei compared to mdx age-matched controls

FIG. 4 is a set of histology slides showing restoration of sarcolemmal stability in SSPN-Tg:mdx muscle.

FIG. 5 is a schematic illustration of protein complexes at the sarcolemma and neuromuscular junction in wild-type, mdx, and SSPN-Tg:mdx muscle.

FIG. 6 is a diagram showing the fiber area of mdx and SSPN-Tg:mdx quadriceps at 6-weeks of age.

### DETAILED DESCRIPTION

The present invention provides compositions and methods in which a patient is treated for a disease involving diminution or dysfunction of a dystrophin-related complex by administering a composition to artificially increase sarcospan activity in a muscle tissue of the patient.

Treatments of all forms of dystrophin-related muscular dystrophy are contemplated, including especially Duchenne's muscular dystrophy. Also contemplated are dystrophin-related muscular atrophies, for example spinal muscular atrophy. The terms “treat”, “treating” and so forth with regard to a patient refer to improving at least one symptom of the subject's disease or disorder. Treating can be curing the disease or condition or improving it, but reducing at least certain symptoms of it.

The term “patient” should be interpreted as including any live human being, or any age, gender, race, nationality, and so forth, regardless of whether that person is or is not currently under the professional care of a physician. The term “providing for administration” should be interpreted as including seeking or obtaining regulatory approval, manufacturing, marketing, selling, re-selling, distributing, prescribing and dispensing, again regardless of whether the channels or trade run though any portion of an official medical establishment.

The term “sarcospan activity in a muscle tissue” should be interpreted herein as whatever activity an extant amount of sarcospan has within the tissue. Defined in that manner, sarcospan is “biologically active” when, for example, sarcospan has an effect on: (a) controlling utrophin protein activity and localization within the myofibre; and/or (b) stabilizing the utrophin-glycoprotein complex. Consistent with this definition, sarcospan activity can be increased, for example, by: (a) directly adding sarcospan and/or or a sarcospan fragment to a tissue; (b) including sarcospan in a composition that is likely to be administered to a tissue; (c) upregulating expression of sarcospan in a tissue; and (d) inhibiting sarcospan degradation.

“Muteins” of sarcospan should be interpreted herein as having at least 30% identity with a native sarcospan, including for example compositions having at least 35%, 40%, 45%, 50%, 55%, 60%, 65%, 70%, 75%, 80%, 85%, 90%, 95% or 98% identity with a native sarcospan (for example with the amino acid sequence set out in SEQ ID NO: 1). Variations from identity can comprise any or more of additions, deletions and substitutions. Contemplated muteins include fragments, truncates and fusion proteins (e.g. comprising fused immunoglobulin, signal sequences or enzyme moieties). Muteins also include proteins in which mutations have been introduced which effectively promote or impair one or more activities of the protein.

Muteins can be produced by any convenient method. Conveniently, site-directed mutagenesis with mutagenic oligonucleotides may be employed using a double stranded template. After verifying each mutant derivative by sequencing, the mutated gene is excised and inserted into a suitable transfection vector so that the modified protein can be over-expressed and purified.

The term “derivative” as applied to sarcospan should be interpreted herein to mean a product of one or more synthetic processes that use a sarcospan, sarcospan fragment or sarcospan mutein as a starting material or reactant. Contemplated derivatives include, for example, fusion proteins (e.g. TAT-tagged proteins), in which the sarcospan, sarcospan fragment or sarcospan mutein has been fused to one or more different proteins or peptides (for example an antibody or a protein domain conferring a biochemical activity, to act as a label, to facilitate cellular uptake or to facilitate purification). Other contemplated derivatives include lipid modified sarcospan proteins.

Sarcospan protein and its fragments can be recombinant or purified from native tissue. The term “sarcospan” includes all native isoforms and variants of human sarcospan. The sequence of the human sarcospan protein is listed below as SEQ ID NO: 1, and a cDNA copy of the mRNA sequence is listed below as SEQ ID NO: 2.

Sarcospan protein fragments may be the most practical means of increasing sarcospan activity. This is based on our discovery that specific regions within sarcospan are responsible for binding the sarcoglycans, and may function to stabilize sarcospan at the muscle plasma membrane. Fragments of all practical lengths and positions are contemplated, including especially fragments having lengths of at least 25, at least 50, and at least 100 amino acids. Preferred sequences for such fragments include amino acid positions 1-53, 54-83, 90-107, 54-83, 90-107, 121-142, 143-187, 188-217, and 218-243 (all with respect to SEQ ID NO: 1, described at Crosbie, R. H., Heighway, J., Venzke, D. P., Lee, J. C. and Campbell, K. P. (1999) “Homo sapiens sarcospan (Kras oncogene-associated gene) (SSPN), mRNA” NCBI Accession No. 005086, see also Table 1 below. These sequences are expected to have significant functionality for binding the sarcoglycans primarily because mutations in these regions disrupt interaction with the sarcoglycans.

TABLE 1

| | |
|---|---|
| 1-53 | Intracellular N-terminus |
| 143-187 | Large extracellular Loop |
| 218-243 | Intracellular C-terminus |
| 54-83; 90-107; | Transmembrane Domains |
| 121-142; 188-217 | |

In another class of embodiments the activity of sarcospan is increased by including in the composition an artificially modified polypeptide including at least a 25 amino acid sequence of a sarcospan protein. Contemplated modifications include a lipid and/or a TAT or other tag.

Any number of lipid modifiers can be selected to enhance targeting of sarcospan to the membrane. Preferred lipid modifications include Ras-derived tags.

Covalent modification of sarcospan with TAT-tags may facilitate entry of sarcospan protein into muscle cells. Although all suitable tags are contemplated, preferred protein transduction tags include both naturally occurring tags (HIV-1 TAT, Ant P penetratin, Vp22 herpes virus) as well as synthetic tags (transportan, Syn B, protegrin peptide) because they are highly efficient cell-penetrating peptides.

Mutations of sarcospan to improve its therapeutic function in muscle are also contemplated. We have shown that specific amino acid regions are important for sarcospan function (Miller et al., 2007). At present, the most preferred mutations are those that increase sarcospan oligomerization, transmembrane domain modifications, and mutations in the large extracellular loop. This is based on the theory/observation that mutations in these regions are critical for stabilization of the dystrophin glycoprotein complex.

Small molecules or proteins are also contemplated to increase endogenous sarcospan expression. For example, the mechanism of sarcospan upregulation may be by altering sarcospan gene activity, stabilizing sarcospan mRNA, or by decreasing sarcospan protein degradation. All practical mechanisms of increasing stability are contemplated, including direct or indirect interaction with sarcospan protein or sarcospan mRNA or sarcospan gene. There are many known protease inhibitors that can be utilized to block sarcospan protein degradation, including for example, BN 82270 (calpain inhibitor), MG-132 (proteosome inhibitor), and Nelfinavir (HIV-1 protease inhibitor).

Regulating sarcospan oligomerization can also provide greater sarcospan protein stability. This is based on our demonstration that sarcospan forms homo-oligomers (Miller et al., 2007; Peter et al., 2007). Various small molecules or polypeptides may function to regulate sarcospan oligomerization, including for example other established tetraspanin, tetraspanin-like, homologous sarcospan proteins. These include, but are not limited to, microspan, nanospan, CD151, CD81, CD9, and the claudins.

Introduction of sarcospan into muscle of patients with muscular dystrophy by means of viral vectors. If necessary, immunosuppressants will be co-administered with virus that encodes sarcospan in case of an adverse immune response from patients. Standard methodology will be used for administration of viral therapies.

Suitable vectors can contain an entire sarcospan gene sequence and/or at least 30 nucleic acids of a sarcospan gene sequence. Preferred mammalian expression vectors contain both prokaryotic sequences to facilitate the propagation of the vector in bacteria, and one or more eukaryotic transcription units that are expressed in eukaryotic cells. These other aspects in the selection, preparation and use of vectors is described in the U.S. Pat. No. 6,864,236 patent, especially from Col. 21/line 41-Col. 25/line 33 are specifically adopted herein.

In yet another class of embodiments the composition comprises a transcription factor or signaling molecules that affect transcription factors that upregulates expression of sarcospan protein. Contemplated transcription factors include MyoD, Mrf, Myf, notch, foxo as well as synthetic transcription factors.

Toxicity and therapeutic efficacy of compounds of the invention can be determined by standard pharmaceutical procedures in cell cultures or experimental animals. The Pharmaceutical compositions of the invention are preferably formulated in accordance with accepted practices and standards. Physiologically acceptable carriers and/or excipients can be used, and different preparations can be formulated, prepared, tested and utilized as suitable for the various route of administration, all in accordance with principles also set forth in the U.S. Pat. No. 6,864,236 patent, especially at Col. 33/line 43-Col. 36/line 16.

In another aspect, the invention contemplates a composition comprising: (a) sarcospan; (b) a biologically active fragment of sarcospan; (c) a biologically active sarcospan mutein; (d) a biologically active sarcospan derivative; (e) nucleic acid encoding any of (a) to (d); or (f) a sarcospan activator, for use in therapy or prophylaxis.

In another aspect, the invention contemplates a pharmaceutical composition comprising: (a) sarcospan; (b) a biologically active fragment of sarcospan; (c) a biologically active sarcospan mutein; (d) a biologically active sarcospan derivative; (e) nucleic acid encoding any of (a) to (d); or (f) a sarcospan activator, together with a pharmaceutically acceptable diluent, excipient or carrier.

In a still further aspect the invention contemplates a composition comprising: (a) sarcospan; (b) a biologically active fragment of sarcospan; (c) a biologically active sarcospan mutein; (d) a biologically active sarcospan derivative; (e) nucleic acid encoding any of (a) to (d); or (f) a sarcospan activator, for use in the treatment of muscular atrophy or muscular dystrophy. In yet another aspect, the invention contemplates the use, for the manufacture of a medicament for the treatment of muscular atrophy or muscular dystrophy, of: (a) sarcospan; (b) a biologically active fragment of sarcospan; (c) a biologically active sarcospan mutein; (d) a biologically active sarcospan derivative; (e) nucleic acid encoding any of (a) to (d); or (f) a sarcospan activator. Sarcospan fragments as hereinabove described are “biologically active” when, for example, the fragments: (a) control utrophin protein activity and localization within the myofibre; and/or (b) stabilize the utrophin-glycoprotein complex.

Theoretical And Experimental Basis

Although not wishing to be held to any particular theory or mechanism(s) of action, the following introductory remarks are intended to provide a theoretical basis underlying the claimed embodiments.

The dystrophin-glycoprotein complex (DGC) is composed of integral and peripheral membrane proteins that span the plasma membrane to connect the extracellular matrix with the intracellular actin cytoskeleton (Campbell and Kahl, 1989; Ervasti and Campbell, 1991; Ervasti and Campbell, 1993; Ervasti et al., 1991; Ervasti et al., 1990; Yoshida and Ozawa, 1990). In skeletal muscle, the DGC provides mechanical stability to the sarcolemma during contraction (Petrof et al., 1993). Mutations in the dystrophin gene are responsible for X-linked Duchenne muscular dystrophy (DMD), which is characterized by progressive wasting of skeletal muscles eventually resulting in cardiac and respiratory failure (for review (Durbeej and Campbell, 2002)). In DMD patients, loss of dystrophin results in absence of the entire DGC complex leading to severe membrane damage and muscle degeneration (for review (Durbeej and Campbell, 2002)). Mdx mice, an established model for DMD, possess a genetic mutation in exon 23 of the murine dystrophin gene, resulting in loss of dystrophin protein. As a result, the DGC is also lost from the sarcolemma, likely due to rapid protein degradation in the absence of a fully assembled complex. Muscles from mdx mice are pathologically similar to DMD patients and display marked membrane disruption due to sarcolemmal instability. Akt signaling is hyper-activated in muscle from DMD patients and mdx mice (Peter and Crosbie, 2006), suggesting that the DGC may also play a role in cellular signaling in addition to its role in mechanical stability of the sarcolemma (Judge et al., 2006).

The transmembrane components of the DGC serve as important anchorage points for organizing the peripheral membrane DGC proteins. These integral membrane proteins include sarcospan (SSPN), the sarcoglycans (γ, β, γ and δ-SG), and β-dystroglycan (β-DG). The SGs and β-DG each possess a single-pass transmembrane domain. Dystrophin, an actin-binding protein, is localized to the subsarcolemma by attachment to the intracellular N-terminus of β-DG (Jung et al., 1995). On the extracellular face of the membrane, β-DG interacts with α-dystroglycan (α-DG) to form a receptor for ligands in the extracellular matrix. The SGs form a tight subcomplex with SSPN (Crosbie et al., 1999). Together, the SG-SSPN subcomplex functions to anchor a-DG attachment to the sarcolemma (Holt and Campbell, 1998). As a whole, the DGC provides a physical linkage across the sarcolemma between the extracellular matrix and the intracellular actin cytoskeleton that functions to protect the membrane from contraction induced damage (for review (Barresi and Campbell, 2006).

It is well-established that stable interactions amongst the integral membrane proteins are critical for DGC function and prevention of muscular dystrophy. Despite their importance, the factors that determine the structural integrity of the DGC are not well understood. The observation that SSPN possesses some sequence homology to the tetraspanin superfamily of proteins raises the possibility that SSPN may serve an important role in mediating and stabilizing protein interactions within the DGC (Crosbie et al., 1997; Crosbie et al., 1999; Crosbie et al., 1998). The tetraspanins each possess four transmembrane domains and function to cluster and organize transmembrane protein complexes, thereby controlling a wide range of cell functions (for review (Hemler, 2003; Levy and Shoham, 2005)). Using a site-directed mutagenesis approach, we have recently shown that SSPN protein exhibits tetraspanin-like properties (Miller et al., 2006). In contrast to the conclusion that SSPN is a non-essential component of the DGC (based on the finding that SSPN-null mice are normal (Lebakken et al., 2000), our recent biochemical studies suggest that SSPN may provide structural information needed for protein organization within the DGC.

As a first test of SSPN function, we generated SSPN transgenic mice (SSPN-Tg) with low (2-3 fold) and moderate (10 fold) levels of SSPN protein over-expression in skeletal muscle (Peter et al., 2007). We found that muscle from SSPN-Tg mice possessed more DGC at the sarcolemma relative to non-transgenic (non-Tg) controls. SSPN was expressed in a nonstoichiometric fashion with respect to the other components of the DGC. Elevation of SSPN disrupted normal interactions within the SG-SSPN subcomplex, which in turn, weakened a-DG attachment to the sarcolemma (Peter et al., 2007). As a result, assembly of the extracellular matrix was disrupted, giving rise to severe congenital muscular dystrophy in mice with moderate levels of SSPN over-expression (Peter et al., 2007). SSPN-Tg mice with low levels of SSPN protein exhibited mild elevation of the DGC, which did not cause any muscle pathology. These findings lead us to conclude that structural elements within SSPN are important for establishing appropriate protein interactions within the DGC. Furthermore, this work lead to the hypothesis that SSPN may function to orchestrate assembly and stability of the DGC.

In order to rigorously test this model of SSPN function, we asked whether SSPN could drive stable assembly of the transmembrane DGC components in dystrophin-deficient muscle. Here, we report that mild (2-3 fold) SSPN over-expression in mdx skeletal muscle does indeed stabilize the SGs and DGs at the sarcolemma thereby preventing premature degradation of these proteins, which normally occurs in the absence of dystrophin. Surprisingly, we found that SSPN over-expression ameliorates the dystrophic pathology, indicative of a functional restoration of the extracellular-intracellular linkage across the sarcolemma. The mechanism of rescue occurs by up-regulation of utrophin, a dystrophin-related protein that is normally restricted to the postsynaptic face of the neuromuscular junction (Khurana et al., 1991; Nguyen et al., 1991; Ohlendieck et al., 1991; Pons et al., 1991). The current report provides the first evidence that SSPN is an important component within the DGC. Furthermore, our findings open exciting and previously unexplored treatment options for Duchenne muscular dystrophy.

Work from SSPN-Tg mice suggests that organization of DG and SG subcomplexes can be regulated, in part, by SSPN (Peter et al., 2007). We rationalized that if SSPN does indeed function as a tetraspanin to coordinate protein interactions, then its expression in dystrophin deficient muscle would anchor the transmembrane components of the DGC within the sarcolemma. In order to test this hypothesis, we engineered transgenic mice to over-express SSPN on the dystrophin-deficient mdx background (SSPN-Tg:mdx). The mdx phenotype is inherited as an X-linked recessive trait, which stems from a premature stop codon in the dystrophin gene, leading to complete absence of dystrophin protein. The DGC is absent from the sarcolemma of mdx muscle likely due to premature degradation of the protein complex in the absence of a fully assembled complex (Ohlendieck and Campbell, 1991). For these studies, we chose to use low (2-3 fold) SSPN over-expressing transgenic mice (SSPN-Tg) lines 31.6 and 29.1, which displayed normal muscle morphology (Peter et al., 2007). SSPN-Tg mice were engineered to carry the human SSPN gene under control of the human skeletal muscle a-actin promoter, which limited SSPN protein expression to skeletal muscles (Peter et al., 2007). Although human and mouse SSPN proteins are over 90% identical at the amino acid level, antibodies specific to human and mouse SSPN permitted us to distinguish between exogenous and endogenous SSPN. SSPN-Tg females were crossed with mdx males to generate dystrophin deficient mice carrying the SSPN transgene (SSPN-Tg:mdx).

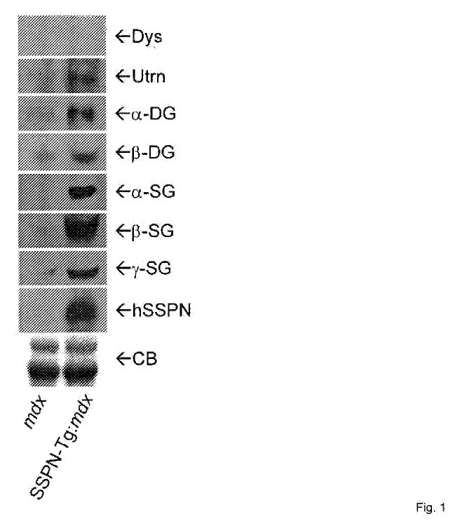

We first determined if SSPN-Tg:mdx muscle produced stable SSPN protein expression and then analyzed how forced SSPN over-expression affected levels of the DGC components in dystrophin-deficient muscle. Total muscle lysates from 6-week old mdx and SSPN-Tg:mdx mice were separated on SDS-PAGE and immunoblotted with antibodies specific to SSPN. Identical blots were probed with antibodies to each of the DGC components. Levels of a-IP-DG as well as a-, p-, and y-SG were dramatically increased in SSPN-Tg:mdx mice relative to mdx controls (FIG. 1) As expected, mdx muscle did not display exogenous (human) SSPN expression. Exogenous SSPN was abundant in SSPN-Tg:mdx muscle samples demonstrating that stable SSPN protein was produced from the transgene (FIG. 1). DG and SG protein expression in SSPN-Tg:mdx muscle was identical to age-matched, non-Tg muscle (data not shown). As expected, dystrophin protein was not detected in samples from either mdx or SSPN-Tg:mdx mice. Surprisingly, we found increased expression of utrophin in SSPN-Tg:mdx muscle relative to mdx muscle (FIG. 1).

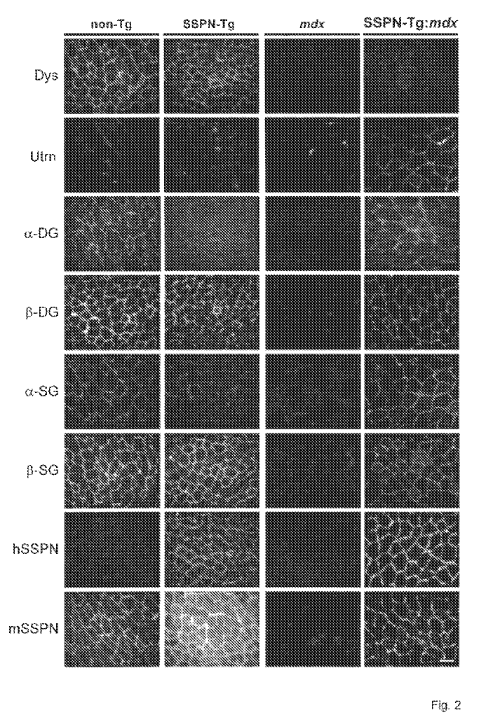

At the postsynaptic region of the neuromuscular junction in wild-type and mdx muscle, utrophin replaces dystrophin to form an otherwise identical utrophin-glycoprotein complex or UGC (Matsumura et al., 1992). Elegant work by Sanes and colleagues has demonstrated that expression and localization of the UGC is important for proper synaptic formation and clustering of acetylcholine receptors at the postsynaptic membrane (Grady et al., 2000). Utrophin is an autosomal homologue of dystrophin and introduction of utrophin in dystrophin-deficient muscle broadens localization of the UGC to the extrasynaptic sarcolemma, where it ameliorates muscular dystrophy (Tinsley et al., 1998; Tinsley et al., 1996). The observation that utrophin is up-regulated in SSPN-Tg:mdx muscle raises the possibility that utrophin and the UGC may localize to the extra-synaptic sarcolemma, where it could functionally replace the DGC. Indirect immunofluorescence was performed on transverse cryosections of SSPN-Tg:mdx quadriceps muscle in order to determine the membrane localization of utrophin and the other components of the UGC. Analysis of non-Tg and SSPN-Tg muscle served as positive controls and mdx muscle was used as a negative control. As shown in FIG. 2, dystrophin expression was robust in positive controls (non-Tg and SSPN-Tg), but was not detected in negative control samples (mdx and SSPN-Tg:mdx). We found that the α-/β-DG as well as a-IP-SG and SSPN, which are not detected in mdx samples, are restored to the sarcolemma in SSPN-Tg:mdx muscle (FIG. 2). Staining of exogenous SSPN (hSSPN) is positive in SSPN-Tg muscles, as expected. Utrophin expression is restricted to the neuromuscular junction in non-Tg, SSPN-Tg, and mdx muscles. However, we find that utrophin displays homogeneous expression at the sarcolemma in SSPNTg:m& muscle, raising the possibility that it forms a functional UGC.

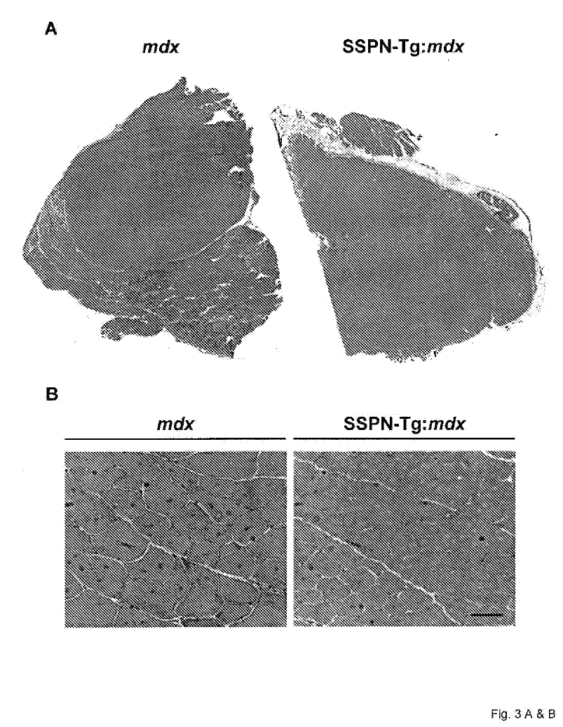

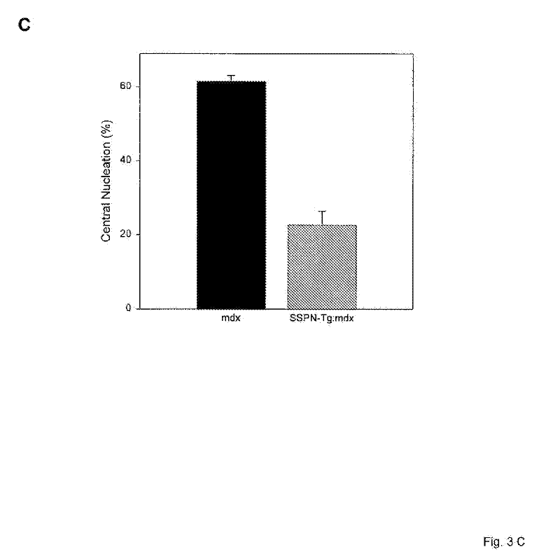

In order to determine if formation of UGC, driven by SSPN over-expression, produced a functional complex, we examined SSPN-Tg:mdx muscle for dystrophic pathology. M& pathology is characterized by progressive muscle degeneration, compensatory hypertrophy, and necrosis of damaged muscle fibers. Histological analysis was performed by staining transverse cryosections of quadriceps muscle with hematoxylin and eosin (H&E). Images of whole quadriceps muscle taken from mdx mice reveal numerous patches of necrosis, which were not present in SSPN-Tg:mdx muscle (FIG. 3A). Central nucleation, a marker of myofiber regeneration, was quantitated by analyzing H&E stained quadriceps muscle at higher magnification. Mdx muscles at 6-weeks of age display dramatic levels (60%) of regenerated myofibers with central nuclei (FIGS. 3B&C). We found a dramatic (40%) reduction in central nucleation in muscle from SSPN-Tg:m& mice (FIGS. 3B&C). Although the level of centrally nucleated fibers in SSPN-Tg:m& mice remains elevated compared to non-Tg controls, this maybe explained by fiber-type specific expression of our transgene (data not shown).

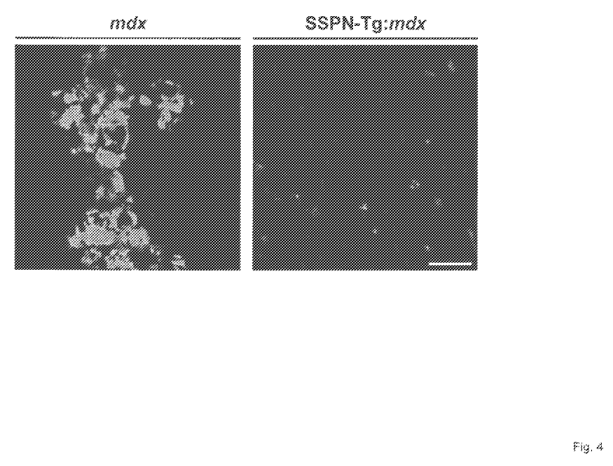

Loss of the DGC from the sarcolemma causes membrane instability, which is a pathological feature of dystrophin-deficient muscular dystrophy (Menke and Jockusch, 1991; Petrof et al., 1993; Straub et al., 1997; Weller et al., 1990). Unrepaired tears in the sarcolemma allow proteins that are normally restricted to the blood serum to freely diffuse across the sarcolemma and accumulate in the sarcoplasm. To further test the functionality of the UGC in SSPN-Tg:mdx mice, we performed a tracer assay with a fluorescent Evans blue dye that binds albumin in blood serum (Straub et al., 1997). Mdx mice display severe sarcolemmal fragility marked by Evans blue dye accumulation in numerous muscle fibers (FIG. 4). In contrast, we never observed Evans blue positive fibers in either SSPN-Tg:mdx or non-Tg muscle (FIG. 4). SSPN is a core component of both the DGC and UGC complexes (Crosbie et al., 1999) and data in the current report demonstrates that mild SSPN protein expression rescues muscular dystrophy in mdx mice by providing stability to the UGC. Davies and colleagues have shown that increased expression of utrophin in mdx mice rescues muscular dystrophy by stabilizing the UGC at the sarcolemma, which restores the structural linkage between the extracellular matrix and the actin cytoskeleton across the membrane bilayer (Tinsley et al., 1998; Tinsley et al., 1996). One therapeutic angle for the treatment of DMD is to identify small molecules that up-regulate utrophin protein expression. We propose that SSPN is a candidate molecule for controlling utrophin protein expression and localization within the myofiber.

Studies have suggested that over-expression of DGC proteins, signaling molecules, and compensatory molecules can improve the pathology of muscle fibers in the mdx mouse. Utrophin, dystrophin, integrin, nNOS, and cytotoxic T-cell GalNAc transferase have been shown to rescue muscular dystrophy. Although these results are incredibly promising, use of these molecules as a truly feasible therapy is debatable. Although utrophin and dystrophin rescue the primary defect of dystrophin-deficiency, genes encoding these proteins are extremely large and require the use of alternatively spliced isoforms for therapeutic adenoviral delivery. Alternatively spliced isoforms may elicit an immune response, which complicates treatment options. Although introduction of GalNAc transferase in dystrophin-deficient mdx mice ameliorates muscular dystrophy, its expression in wild-type mice is toxic, which poses obvious problems in using this as a therapy.

SSPN is unique and offers numerous advantages over current therapeutic strategies. The small size of SSPN makes it a very attractive for gene delivery. The SSPN cDNA is under 1-kb, which is well within the range for easy packaging into adeno-associated viral vectors for delivery. Often, gene therapies involving large genes require truncation of the cDNA in order to be inserted into the limited viral genome. The fact that full-length SSPN can be easily packaged into viral vectors circumvents the necessity to generate recombinant forms of SSPN (which is the case for dystrophin and utrophin). This is important because recombinant proteins may trigger an immune response, which could be lethal. Because SSPN is expressed in a variety of nonmuscle tissues, even in dystrophin-deficiency, increasing the expression of SSPN in skeletal muscle should not pose an immune threat.

The results in the current study were unexpected because, for nearly ten years, the precise function of SSPN has remained elusive and assumed to be inconsequential. However, our studies show that SSPN is a critical mediator of protein interactions within the DGC and UGC. Furthermore, SSPN prevents muscular dystrophy by coordinating assembly, targeting, and stability of the UGC. This is impressive considering that SSPN, the smallest member of the complex, must orchestrate proper structural arrangement of over seven large, peripheral and integral membrane proteins. These properties are singularly unique to SSPN, making SSPN the ‘underdog’ component of the DGC.

Generation of SSPN-Tg:mdx Mice

Transgenic constructs were engineered with the human skeletal actin promoter and the VP1 intron upstream of human SSPN, as described previously ((Crawford et al., 2000; Peter et al., 2007; Spencer et al., 2002)). SSPN transgenic males, line (29.1), were bred with mdx females (Jackson Laboratories, Bar Harbor, Me.) to produce male SSPN-Tg:m& mice. Female SSPN-Tg and non-Tg mice as well as male non-Tg:mdx mice were utilized as controls. Mice were analyzed at 6-weeks-of-age. Transgenic mice used for this study expressed an approximately 2-fold increase in SSPN protein expression in skeletal muscle compared to female non-Tg mice (Peter et al., 2007). All mice were maintained in the Life Sciences Vivarium and all procedures were carried in accordance with guidelines set by the UCLA Institutional Animal Care and Use Committee.

Immunofluorescence

Quadriceps from female SSPN-Tg, female non-Tg, male non-Tg:mdx, and SSPN-Tg:mdx mice were dissected from 6-week-old mice. Muscles were covered in 10.2% polyvinyl alcohoV4.3% polyethylene glycol and rapidly frozen in liquid nitrogen cooled isopentane. Mounted muscle was stored at −80° C. until analyzed. Transverse sections (8 μm) were prepared utilizing a Leica CM 3050s cryostat (Leica Microsystems, Bannockburn, Ill.) and stored on positively charged glass slides (VWR, West Chester, Pa.). Sections were stored at −80° C. for future analysis. Sections were acclimated to RT for 15 min and then blocked using 3% BSA diluted in PBS for 1 hr at RT. Sections were then incubated at 4° C. for 18 hrs with antibodies to the following proteins (antibody dilutions are indicated): dystrophin (University of Iowa, Hybridoma Facility, Iowa City, Iowa; MANDYS 1, 1:10), utrophin (Vector Laboratories, Burlingame, Calif.; NCL-DRP2 1:5), α-DG (Upstate Signaling, Lake Placid, N.Y.; VLA4-1, 1:100), β-DG (University of Iowa, Hybridoma Facility; MANDAG2, 1:50), α-SG (Vector Laboratories; VP-AlO5, 1:100), β-SG (Vector Laboratories; VP-B206, 1:50), human SSPN (affinity purified Rabbit 15, 1:25), and mouse SSPN (affinity purified Rabbit 18, 1:25). Polyclonal antibodies to endogenous (mouse) and exogenous (human) SSPN have been described previously. Primary antibodies were detected by FITC conjugated anti-rabbit and anti-mouse (Jackson ImmunoResearch, West Grove, Pa.) secondary antibodies diluted at 1:500. Secondary antibodies were incubated for 1 hr at RT. To preserve fluorescence signal, sections were mounted in VectaShield (Vector Laboratories). To determine the level of non-specific staining, secondary antibodies alone were incubated with sections. Mounted sections were visualized using the Axioplan 2 fluorescent microscope equipped with a Plan-neofluar 40xf1.3 oil differential interference contrast objective (Carl Zeiss Inc, Thornwood, N.Y.), and images were captured utilizing Axiovision 3.0 software (Carl Zeiss Inc).

Evans Blue Tracer Assay

In order to establish sarcolemmal integrity, mice were injected with 50 pl of Evans Blue Dye (10 mg/ml in sterile 10 mM phosphate buffer, 150 mM NaCI, pH 7.4) per 10 g of body weight as described previously (Straub et al., 1997). Peritoneal cavity injection was performed on 6-week old SSPN-Tg:mdx and non-Tg:mdx mice. 24 hours post injection, quadriceps were excised and mounted as described above. Transverse sections (8 pm) were briefly fixed in ice-cold acetone, washed in PBS (10 mM phosphate buffer, 150 mM NaCI, pH 7.4), and mounted with Vectashield (Vector Laboratories). Evans blue positive myofibers were observed using an Axioplan 2 fluorescent microscope and a Plan-neofluar 40˜11.3 oil differential interference contrast objective (Carl Zeiss Inc).

Histology

Hematoxylin and eosin (H&E) staining was utilized for visualization of fibrosis, central nuclei, and fiber diameter. Transverse quadriceps sections (8 μm) were left at RT for 15 min before beginning staining procedure. Sections were incubated with hematoxylin for 3 min, washed with water for 1 min, incubated with eosin for 3 min, dehydrated in solutions of 70%, 80%, 90% and 100% ethanol. Sections were then incubated in xylene for a total of 6 min. Sections were left at RT briefly to dry before mounting with Permount. All supplies for the H&E staining were purchased from Fisher Scientific (Fairlawn, N.J.). To measure the fiber area and count centrally nucleated fibers, digitized images were captured under identical conditions using an Axioplan 2 fluorescent microscope and Axiovision 3.0 software (Carl Zeiss Inc). Percentage of centrally nucleated fibers and fiber areas were assessed for 4 SSPN-Tg:mdx and 4 non-Tg:mdx mice. Data for central nucleation is represented as percentage of total fibers counted. Fiber areas and central nucleation percentages were averaged and the standard error is represented. Sigma Plot® (Systat Software Inc, Point Richmond, Calif.) was utilized to perform statistical analysis.

Protein Preparation

Skeletal muscle was snap frozen in liquid nitrogen and ground to a fine powder with a mortar and pestle. Pulverized muscle was added to ice-cold modified RIPA lysis buffer with phosphatase inhibitors (1% Nonidet P-40, 0.5% sodium deoxycholate, 0.1% SDS, 1 mM EDTA, 5 mM N-ethylmaleimide, 50 mM sodium fluoride, 2 mM β-glycerophosphate, 1 mM sodium orthovanadate, 100 nM okadaic acid, 5 nM microcystin LR, and 20 mM Tris-HC1, pH 7.6) and protease inhibitors (0.6 μg/mL pepstatin A, 0.5 μg/mL aprotinin, 0.5 μg/mL leupeptin, 0.75 mM benzamidine, and 0.1 mM PMSF). Homogenates were rocked at 4 OC for 1 hour. Following clarification by centrifugation at 15,000×g for 15 min, tissue lysates were stored at −80° C.

Immunoblot Analysis

Protein concentrations were determined using the DC Protein Assay® (Bio-Rad, Hercules, Calif.). Equal concentrations (60 μg) of protein samples were resolved by 5%, 10%, or 15% SDS-PAGE and transferred to nitrocellulose membranes (Millipore Corp., Billerica, Mass.) for subsequent immunoblotting. Primary antibodies against dystrophin (University of Iowa, Hybridoma Bank; MANDY S 1, 1:10), utrophin (Vector Laboratories; NCL-DRP2, 1:5), a-DG (Upstate Signaling; IIH6C-4, 1:1000), P-DG (University of Iowa, Hybridoma Bank; MANDAG2, 1:250), a-SG (Vector Laboratories; VP-A105, 1:1 OO), P-SG (Vector Laboratories; VP-B206, 1:100), γ-SG (Vector Laboratories; VP-G803, 1:100), and human SSPN (affinity purified Rabbit 15, 1:500) were incubated at RT while shaking for 18 hours. Horseradish peroxidase-conjugated antirabbit IgG (Amersham Pharmacia Biotech, Piscataway, N.J.) and anti-mouse IgG (Amersham Pharmacia Biotech) and IgM (Upstate Signaling) secondary antibodies were used at a 1:3,000 dilution. All immunoblots were developed using enhanced chemiluminescence (Supersignal West Pico Chemiluminescent substrate, Pierce, Rockford, Ill.).

### ABBREVIATIONS

DG, dystroglycan

DGC, dystrophin-glycoprotein complex

mdx, dystrophin-deficient mice

SG, sarcoglycan

SSPN, sarcospan

Tg, transgene

UGC, utrophin-glycoprotein complex

### REFERENCES

Barresi, R., and K. P. Campbell. 2006. Dystroglycan: from biosynthesis to pathogenesis of human disease. J Cell Sci. 119:199-207.
Campbell, K. P., and S. D. Kahl. 1989. Association of dystrophin and an integral membrane glycoprotein. Nature. 338:259-62.
Crawford, G. E., J. A. Faulkner, R. H. Crosbie, K. P. Campbell, S. C. Froehner, and J. S. Chamberlain. 2000. Assembly of the dystrophin-associated protein complex does not require the dystrophin COOH-terminal domain. J Cell Biol. 150:1399-4 10.
Crosbie, R. H., J. Heighway, D. P. Venzke, J. C. Lee, and K. P. Campbell. 1997. Sarcospan: the 25 kDa transmembrane component of the dystrophin-glycoprotein complex. J. Biol. Chem. 272:3 1221-3 1224.
Crosbie, R. H., C. S. Lebakken, K. H. Holt, D. P. Venzke, V. Straub, J. C. Lee, R. M. Grady, J. S. Chamberlain, J. R. Sanes, and K. P. Campbell. 1999. Membrane targeting and stabilization of sarcospan is mediated by the sarcoglycan subcomplex. J Cell Biol. 145:153-65.
Crosbie, R. H., H. Yamada, D. P. Venzke, M. P. Lisanti, and K. P. Campbell. 1998. Caveolin-3 is not an integral component of the dystrophin-glycoprotein complex. FEBS Letters. 427:279-282.
Durbeej, M., and K. P. Campbell. 2002. Muscular dystrophies involving the dystrophinglycoprotein complex: an overview of current mouse models. Curr Opin Genet Dev. 12:349-61.
Ervasti, J. M., and K. P. Campbell. 1991. Membrane organization of the dystrophin-glycoprotein complex. Cell. 66:1 12 1-3 1.
Ervasti, J. M., and K. P. Campbell. 1993. A role for the dystrophin-glycoprotein complex as a transmembrane linker between laminin and actin. Journal of Cell Biology. 122:809-23.
Ervasti, J. M., S. D. Kahl, and K. P. Campbell. 1991. Purification of dystrophin from skeletal muscle. Journal of Biological Chemistry. 266:9161-5.
Ervasti, J. M., K. Ohlendieck, S. D. Kahl, M. G. Gaver, and K. P. Campbell. 1990. Deficiency of a glycoprotein component of the dystrophin complex in dystrophic muscle. Nature. 345:315-9.
Grady, R. M., H. Zhou, J. M. Cunningham, M. D. Henry, K. P. Campbell, and J. R. Sanes. 2000. Maturation and maintenance of the neuromuscular synapse: genetic evidence for roles of the dystrophin-glycoprotein complex. Neuron. 25:279-93.
Hemler, M. E. 2003. Tetraspanin proteins mediate cellular penetration, invasion, and fusion events and define a novel type of membrane microdomain. Annu Rev Cell Dev Biol. 19:397-422.
Holt, K. H., and K. P. Campbell. 1998. Assembly of the sarcoglycan complex. Insights for muscular dystrophy. J. Biol Chem. 273:34667-70.
Hoyte, K., V. Jayasinha, B. Xia, and P. T. Martin. 2004. Transgenic overexpression of dystroglycan does not inhibit muscular dystrophy in mdx mice. Am J Pathol. 164:7 1 1-8.
Judge, L. M., M. Haraguchiln, and J. S. Chamberlain. 2006. Dissecting the signaling and mechanical functions of the dystrophin-glycoprotein complex. J Cell Sci. 1 19:1537-46.
Jung, D., B. Yang, J. Meyer, J. S. Chamberlain, and K. P. Campbell. 1995. Identification and characterization of the dystrophin anchoring site on beta-dystroglycan. Journal of Biological Chemistry. 270:27305-10.
Khurana, T. S., S. C. Watkins, P. Chafey, J. Chelly, F. M. Tome, M. Fardeau, J. C. Kaplan, and L. M. Kunkel. 1991. Immunolocalization and developmental expression of dystrophin related protein in skeletal muscle. Neuromuscular Disorders. 1:185-94.
Krag, T. O. B., Grd-Hansen, M., Khurana, T. S. 2001. Harnessing the potential of dystrophin-related proteins for ameliorating Duchenne's muscular dystrophy, Acta Physiologica Scandinavica, 171:349-358.
Lebakken, C. S., D. P. Venzke, R. F. Hrstka, C. M. Consolino, J. A. Faulkner, R. A. Williamson, and K. P. Campbell. 2000. Sarcospan-deficient mice maintain normal muscle function. Mol Cell Biol. 20:1669-77.
Levy, S., and T. Shoham. 2005. The tetraspanin web modulates immune-signalling complexes. Nut Rev Immunol. 5:136-48.
Matsumura, K., J. M. Ervasti, K. Ohlendieck, S. D. Kahl, and K. P. Campbell. 1992. Association of dystrophin-related protein with dystrophin-associated proteins in mdx mouse muscle. Nature. 360588-91.
Menke, A., and H. Jockusch. 1991. Decreased osmotic stability of dystrophin-less muscle cells from the mdx mouse. Nature. 349:69-71.
Miller, G., E. L. Wang, K. L. Nassar, A. K. Peter, and R. H. Crosbie. 2006. Structural and Functional Analysis of the Sarcoglycan-Sarcospan Subcomplex. Exp Cell Res in press.
Nguyen, T. M., J. M. Ellis, D. R. Love, K. E. Davies, K. C. Gatter, G. Dickson, and G. E. Morris. 1991. Localization of the DMDL gene-encoded dystrophin-related protein using a panel of nineteen monoclonal antibodies: presence at neuromuscular junctions, in the sarcolemma of dystrophic skeletal muscle, in vascular and other smooth muscles, and in proliferating brain cell lines. Journal of Cell Biology. 115:1695-700.
Ohlendieck, K., and K. P. Campbell. 1991. Dystrophin-associated proteins are greatly reduced in skeletal muscle from mdx mice. Journal of Cell Biology. 1 15:1685-94.
Ohlendieck, K., J. M. Ervasti, K. Matsumura, S. D. Kahl, C. J. Leveille, and K. P. Campbell. 1991. Dystrophin-related protein is localized to neuromuscular junctions of adult skeletal muscle. Neuron. 7:499-508.
Peter, A. K., and R. H. Crosbie. 2006. Hypertrophic response of Duchenne and limb-girdle muscular dystrophies is associated with activation of Akt pathway. Exp Cell Res. 312:2580-91.
Peter, A. K., G. Miller, and R. H. Crosbie. 2007. Disrupted mechanical stability of the dystrophinglycoprotein complex causes severe muscular dystrophy in sarcospan transgenic mice. JCS in press.
Petrof, B. J., J. B. Shrager, H. H. Stedman, A. M. Kelly, and H. L. Sweeney. 1993. Dystrophin protects the sarcolemma from stresses developed during muscle contraction. Proceedings of the National Academy of Sciences of the United States of America. 90:3710-4.
Pons, F., N. Augier, J. O. Leger, A. Robert, F. M. Tome, M. Fardeau, T. Voit, L. V. Nicholson, D. Mornet, and J. J. Leger. 1991. A homologue of dystrophin is expressed at the neuromuscular junctions of normal individuals and DMD patients, and of normal and mdx mice. Immunological evidence. FEBS Letters. 282:161-5.
Spencer, M. J., J. R. Guyon, H. Sorimachi, A. Potts, I. Richard, M. Herasse, J. Chamberlain, I. Dalkilic, L. M. Kunkel, and J. S. Beckmann. 2002. Stable expression of calpain 3 from a muscle transgene in vivo: immature muscle in transgenic mice suggests a role for calpain 3 in muscle maturation. Proc Natl Acad Sci USA. 99:8874-9.
Straub, V., J. A. Rafael, J. S. Chamberlain, and K. P. Campbell. 1997. Animal models for muscular dystrophy show different patterns of sarcolemmal disruption. J Cell Biol. 139:375-385.
Tinsley, J., N. Deconinck, R. Risher, D. Kahn, S. Phelps, J. M. Gillis, and K. Davies. 1998. Expression of full-length utrophin prevents muscular dystrophy in mdx mice. Nut. Med 4:1441-1444.
Tinsley, J. M., A. C. Potter, S. R. Phelps, R. Fisher, J. I. Trickett, and K. E. Davies. 1996. Amelioration of the dystrophic phenotype of mdx mice using a truncated utrophin transgene. Nature. 384:349-353.
Weller, B., G. Karpati, and S. Carpenter. 1990. Dystrophin-deficient mdx muscle fibers are preferentially vulnerable to necrosis induced by experimental lengthening contractions. J Neurol Sci. 100:9-13.
Yoshida, M., and E. Ozawa. 1990. Glycoprotein complex anchoring dystrophin to sarcolemma. JBC. 108:748-52.

### FIGURE LEGENDS

FIG. 1. SSPN over-expression increases expression of UGC in mdx mice. Skeletal muscle from mdx and SSPN-Tg:mdx tissue was solubilized in modified RIPA buffer and protein samples (60 μg) were resolved by SDS-PAGE and immunoblotted using antibodies against dystrophin (Dys), utrophin (Utrn), dystroglycan (a- and β-DG), exogenous SSPN (hSSPN) as well as the sarcoglycans (α, β, and γ-SG). As expected, hSSPN was only detected in SSPN-Tg:mdx muscle. Expression levels of DGs and SGs were dramatically elevated in SSPN-Tg:mdx muscle samples. Utrophin, a homolog of dystrophin, is up-regulated in SSPNTg: mdx tissue. Equal loading of mdx and SSPN-Tg:mdx lysates was confirmed by Coomassie blue (CB) staining. Molecular weights are indicated (×10³ Da).

FIG. 2. SSPN stabilizes the UGC at the sarcolemma. Transverse cryosections of quadriceps muscle from non-Tg, SSPN-Tg, mdx, and SSPN-Tg:mdx mice were stained with the antibodies to dystrophin (Dys), utrophin (Utrn), dystroglycan (α and β-DG), mouse SSPN (mSSPN), and transgenic human SSPN (hSSPN) as well as the sarcoglycans (α. β, and γ-SG). Protein staining was visualized by indirect immunofluorescence. Bar, 100 μm.

FIG. 3. SSPN-Tg:mdx mice display near normal muscle pathology. (A) Transverse cryosections of whole quadriceps muscle from age-matched mdx and SSPNTg: mdx mice were stained with hematoxylin and eosin (H&E) to visualize muscle pathology. Many necrotic patches (asterisk) are visible in mdx quadriceps. Necrotic fibers were never observed in SSPN-Tg:mdx quadriceps. (B) H&E staining of quadriceps muscle from mdx and SSPN-Tg:mdx mice is shown at higher magnification. Note the presence of numerous myofibers with central nucleation as well as the variation in fiber size evident in mdx tissue. Muscle from SSPN-Tg:mdx muscle displays reduced central nucleation and more uniform fiber size. Bar, 100 μm. (C) Central nucleation (% of total fibers) was quantified for quadriceps isolated from mdx and SSPN-Tg:mdx (n=4). SSPN-Tg:mdx mice display a 3-fold reduction in central nuclei compared to mdx age-matched controls. Each value represents mean ±SEM of total quadriceps analyzed (*P=6.0×10⁻⁴).

FIG. 4. Sarcolemmal stability is restored in SSPN-Tg:mdx muscle. To examine infiltration of blood serum proteins into damaged muscle fibers, mdx and SSPN-Tg: mdx mice were intraperitoneally injected with Evans blue dye, a marker for membrane instability. Mdx quadriceps displays many Evans blue dye positive fibers (visualized by red fluorescence), which is a marker for membrane damage. Evans blue dye was not detected in muscle from SSPN-Tg:mdx mice, demonstrating that SSPN expression restored membrane stability in dystrophin-deficient muscle. Bar, 20 μm.

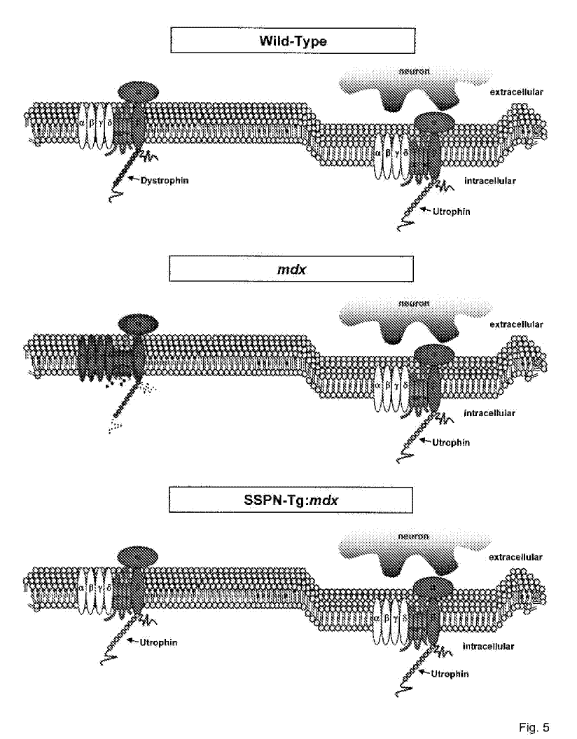

FIG. 5: Model of SSPN-mediated rescue of dystrophin-deficient muscular dystrophy. Schematic illustration of protein complexes at the sarcolemma and neuromuscular junction in wild-type, mdx, and SSPN-Tg:mdx muscle. In wild-type tissue, dystrophin (black), the DGs (red), SSPN (green), and the SGs (yellow) form the DGC. Utrophin (blue) forms an analogous complex, the UGC, at the neuromuscular junction in skeletal muscle. In dystrophin-deficient tissue, the UGC remains at the neuromuscular junction. However, due to the loss of dystrophin, the DGs, SSPN, and the SGs are absent from the sarcolemma. It is unclear if the components of the DGC are separately transported to the sarcolemma, assembles, and degrades if the interaction with dystrophin fails to occur or if the complex assembles in the cytoplasm and degrades in the absence of dystrophin before reaching the sarcolemma. SSPN over-expression in dystrophin deficient tissue restores sarcolemmal integrity by readdressing the UGC to the sarcolemma. Because little is known about the assembly of the DGs, the SGs, and SSPN, the mechanism of UGC translocation to the sarcolemma remains unclear. If each of the components is transported separately to the sarcolemma, SSPN may act as a stabilizer of the DGs and the SGs allowing utrophin to interact with the components at the sarcolemma. Alternatively, SSPN may have a higher affinity for utrophin-associated protein complexes and may mediate protein interactions within the cytosol before the complexes are separately translocated to the sarcolemma and neuromuscular junction. The ability of SSPN to function as an anchor for the other transmembrane components of the DGC is a property that is unique to SSPN. Over-expression of other DGC components, including DG and γ-SG, in mdx muscle did not facilitate stability of the complex at the sarcolemma (Hoyte et al., 2004).

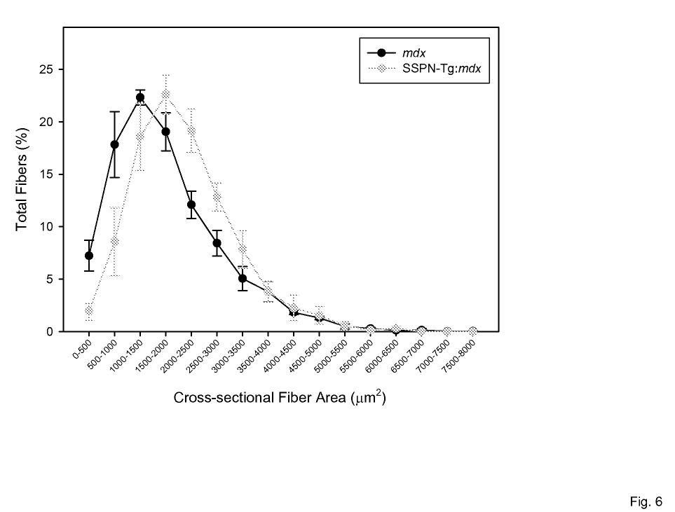

FIG. 6: Reduction of atrophic fibers upon SSPN over-expression. Skeletal muscle atrophy is reduced upon over-expression of SSPN. Fiber cross-sectional areas were quantitated for quadriceps isolated from mdx and SSPN-Tg:mdx (n=4). There is a shift to larger fiber areas in SSPN-Tg:mdx mice indicating reduction in atrophy in fibers over-expressing SSPN. For each data point, mean ±SEM is shown.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of treating a disease related to diminution or dysfunction of a dystrophin-related complex in a patient, comprising providing for administration to the patient, a composition to artificially increase sarcospan activity in a muscle tissue of the patient.

2. The method of claim 1, wherein the composition comprises a complete sarcospan protein.

3. The method of claim 1, wherein the composition comprises a sarcospan protein fragment having a length of at least 25 amino acids.

4. The method of claim 1, wherein the composition comprises a lipid modified polypeptide including at least a 25 amino acid sequence of a sarcospan protein.

5. The method of claim 1, wherein the composition comprises a TAT-tagged polypeptide including at least a 25 amino acid sequence of a sarcospan protein.

6. The method of claim 1, wherein the composition comprises an artificially modified sarcospan protein.

7. The method of claim 1, wherein the composition comprises a sarcospan protein degradation inhibitor.

8. The method of claim 1, wherein the composition comprises a sarcospan oligomerization inhibitor.

9. The method of claim 1, wherein the composition comprises a vector that includes a construct having at least a 30-mer fragment of a sarcospan gene sequence.

10. The method of claim 1, wherein the composition comprises a vector that includes an entire sarcospan gene sequence.

11. The method of claim 1, wherein the composition comprises a transcription factor that upregulates expression of the sarcospan.

12. The method of claim 1, wherein the disease is selected from the list consisting of muscular dystrophy and muscular atrophy.

13. An artificially modified polypeptide including at least a 25 amino acid sequence of a sarcospan protein.

14. The peptide of claim 13 wherein the modification comprises addition of a targeting component selected from a membrane targeting lipid and a cell-penetrating peptide.

15. The peptide of claim 13 wherein the modification comprises a stabilizer.

16. The peptide of claim 13 wherein the modification comprises a degradation inhibitor.

17. The peptide of claim 13 wherein the modification comprises an oligomerization inhibitor.

18. A transfection vector having at least a 30-mer fragment of a sarcospan gene sequence.

19. A transfection vector having a complete sarcospan gene sequence.
